(19) United States
(12) Patent Application Publication
(10) Pub. No.: US 2023/0241125 A1
(43) Pub. Date: Aug. 3, 2023

(54) HONEY POWDER HAVING HIGH HONEY AND ANTIOXIDANT CONTENT AND PRODUCTION OF IT

(71) Applicant: SBS BILIMSEL BIO ÇÖZÜMLER SANAYI VE TICARET A.S., Istanbul (TR)

(72) Inventors: Asli Elif TANUGUR SAMANCI, Istanbul (TR); Taylan SAMANCI, Istanbul (TR)

(21) Appl. No.: 18/003,422

(22) PCT Filed: Dec. 12, 2020
(86) PCT No.: PCT/TR2020/051288

§ 371 Date: Dec. 27, 2022

(30) Foreign Application Priority Data
Jul. 3, 2020 (TR) 2020/10568

(51) Int. Cl. A61K 35/644; A61K 9/16; A23L 21/25; A23L 29/30; A23L 29/212; A23L 29/281; A23L 29/231; A23L 33/17; A23L 33/105; A23L 27/00

(52) U.S. Cl. CPC A61K 35/644; A23L 21/25; A23L 27/72; A23L 29/35; A23L 29/212; A23L 29/231; A23L 29/284; A23L 33/17; A23L 33/105; A61K 9/1652; A61K 9/1658; A61K 9/1664; A61K 9/1694

(57) ABSTRACT

Disclosed is a method for production of honey powder that includes the process steps of mixture of at least an auxiliary encapsulation agent selected from a group consisting of water soluble gum, maltodextrin, starch, gelatine, sodium caseinate pectin or mixture of them, measuring viscosity of mixture, adding distilled water to mixture until a viscosity value of 15-20 mPa.s is achieved, applying a homogenization process, bringing a temperature of mixture to 45° C., and obtaining honey powder by drying at a spray drier.

### TECHNICAL FIELD

Invention relates to honey powder products.

Invention particularly relates to honey powder of natural sources, not containing chemical additives and usable as raw material or directly as a product in food sector for healthy nutrition purposes, and a method for production of it.

### PRESENT STATE OF THE ART

Glucose-fructose syrup, saccharine or other chemical sweeteners are used as sweetening agent in ice cream, biscuits, candies, chocolate, and other products in food sector. Although these ingredients improve taste of the final product and give energy to consumer, they are poor in terms of nutritional richness.

Another sweetener used in food products is honey. It is a good sweetener alternative due to the sugar concentration and fragrance it contains. Sugar composition of honey comprises about 25 types of oligosaccharide (disaccharides, trisaccharides, tetrasaccharides), mainly glucose and fructose. In addition, it is considerably rich nutrient in terms of B1, B2, B3, B5, B6 vitamins. For that reason, honey is a strong antiseptic and has antibacterial feature. It heals or protects from various diseases because of this feature. Because of all those reasons, it is a significant sweetener.

However, because of its high viscosity, honey is in semi-fluid form in natural form. This is a feature makes it difficult to process honey. For that reason, in order to use honey as a raw material in food products, it is preferred to reduce the moisture content by drying and to make it powder. In addition, dried form of honey can be used as raw material as well as consumed easily as a product itself. Under the name of honey powder, some products containing little or no honey, sugar syrup, honey flavor and chemical additives are used in the market. Such products and derivatives can be used as raw material and also products in honey powder form are not available. In addition, to extend shelf life, it contains several various chemical additives. Such chemical additives can be harmful for human health. Since these products do not contain honey, they mislead the consumer and prevent the use of honey produced in our country in the food industry.

Honey powder with low moisture content is more advantageous than liquid honey with many features such as being easier to transport, needing less storage space, ease of processing and longer shelf life. However, several problems are encountered in drying honey because of high sugar content. Another problem is the sticking and caking due to sugar such as fructose in its content. There are studies focusing on processes preventing all of those.

National patent application numbered TR2009/08388 encountered in the literature relates to method for production of dry honey powder. Spray drier or cylindrical drier is used in honey powder production. In both methods, maltodextrin and silicone dioxide additive agents are added appropriately and the obtained mixture is given to said devices and honey powder is obtained.

As a result, due to above described disadvantages and inadequacy of existing solutions it has been necessary to make development in the related art.

### BRIEF DESCRIPTION OF THE INVENTION

The present invention relates to honey powder meeting the needs mentioned above, eliminating all disadvantages and providing some additional advantages, and a manufacturing method for the powder.

The primary purpose of the invention is to produce honey powder with the desired moisture content, which preserves its natural aroma and nutritional properties, and has sweetening properties. Another purpose of the invention is to provide a honey powder not having side effects or medicine-food interaction, and production of such powder.

A further purpose of the invention is the honey powder and the production of this powder, which enables the high moisture content honey produced in our country to be evaluated as new value-added products. In order to achieve above mentioned purposes, the invention is a honey powder and comprises at least an Auxiliary capsulation agent selected from a group consisting of water soluble gum, maltodextrin, starch, gelatine, sodium caseinate pectin or mixture of them. The invention also covers use of honey powder for healthy diet or apitherapy purpose.

In order to achieve above mentioned purposes, the invention is a method for production of honey powder and comprises process steps of mixture of at least a auxiliary encapsulation agent selected from a group consisting of water soluble gum, malto-dextrin, starch, gelatine, sodium caseinate pectin or mixture of them, measuring viscosity of mixture, Adding distilled water to mixture until value of 15-20 mPa.s is achieved, to mixture until viscosity value of 15-20 mPa.s is achieved, applying homogenization process, bringing temperature of mixture to 45° C., obtaining honey powder by drying at spray drier.

The structural and characteristics features of the invention and all advantages will be understood better in detailed descriptions given below and therefore, the assessment should be made taking into account the detailed explanations.

### DETAILED DESCRIPTION OF THE INVENTION

In this detailed description, the honey powder of the invention and the production method of this powder are explained only for a better understanding of the subject and without any limiting effect. Honey powder of invention in the most basic form comprises at least an auxiliary encapsulation agent selected from a group consisting of water soluble gum, malto-dextrin, starch, gelatine, sodium caseinate pectin or mixture of them. Said auxiliary encapsulation agent is preferably in 50 % - 50 % mixture and more preferably a water soluble gum or maltodextrin or mixture thereof.

Honey; is a natural product that plant nectars, secretions in the living parts of the plants or the secretions of plant sucking insects living on the living parts of the plants are combined with specific substances after they are collected by the honey bee, reduce the water content and mature by storing with honeycomb.

Water soluble gum; Water Soluble Gum is sticky, gummy, natural substances that leak from plants They are defined as water soluble or dispersible polymeric carbohydrates used to give thickening agent and/or gel effect. In this invention, it can be used as auxiliary encapsulation agent to obtain honey powder product.

Maltodextrin; is obtained commercially by partly breaking down starch. It is used as thickening giving agent in food industry. Maltodextrin which is white, low sweetening level powder displays very high viscosity as solvent despite not keeping water alone. Due to its indefinite neutral taste, it does not affect other food flavors, easily disperses in cold and water-based systems, and exhibits a structure that can preserve its clarity and visual appearance.

Starch; is an essential nutrient. Flour made from wheat, foods made from flour, mainly bread constitutes most part of our daily foods. In addition, potato. Rice, corn are also mostly starch nutrients. In this invention, it can be used as auxiliary encapsulation agent providing production of honey powder product.

Gelatine; is a colourless, odourless, thickening enhancer used in food products generally and made from bone and cartilage or plant algae. In this invention, it can be used as auxiliary encapsulation agent providing production of honey powder product.

Sodium caseinate; is used in food and medicine products requiring high emulsification and fast dispersion. It offers high nutrition for food and medicine products. It is a sodium containing salt compound of casein which is an essential dairy protein. In this invention, it can be used as auxiliary encapsulation agent providing production of honey powder product.

Pectin; is the major binding component of the cell walls of plants and fruits. Pectin has feature of forming gel together with sugar. For that reason, pectin is used in combination of sugar as thickening agent in food industry. In our invention, it is used as auxiliary encapsulation agent providing production of powder product.

In an illustrative alternative of the invention, honey powder preferably comprises natural protein, fruit juice concentrate or mixture thereof. Natural protein is big molecules containing amino acid and needed for proper functioning of our body and all cells in our body. Protein is a compound strengthening muscles, preventing anaemia, keeping body strength high. Fruit juice concentrate is a product obtained by means of dewatering fruit juice of one or more than one fruit by physical means at certain rates.

Table 1 shows preferred contents of honey powder of the invention and quantities thereof. Product developed under the invention is used for food, healthy feeding and apitherapy purposes.

TABLE 1

Raw materials used in the method of the invention

| Description of Component | Preferred amount (gr | Usable amount (gr) | Preferred amount (%) | Usable amount (%) |
|---|---|---|---|---|
| Honey | 500 | 250 - 750 | 48,07 | 46,07 - 50,07 |
| Natural Protein | 20 | 10-50 | 1,92 | 0,92- 2,92 |
| Water Soluble Gum | 500 | 250 - 750 | 48,07 | 46,07 - 50,07 |
| Maltodextrin | 500 | 250 - 750 | 48,07 | 46,07 - 50,07 |
| Fruit juice concentrate | 20 | 10-50 | 1,92 | 0,92- 2,92 |
| Starch | 500 | 250 - 750 | 48,07 | 46,07 - 50,07 |
| Gelatine | 500 | 250 - 750 | 48,07 | 46,07 - 50,07 |
| Sodium caseinate | 500 | 250 - 750 | 48,07 | 46,07 - 50,07 |
| Pectin | 500 | 250 - 750 | 48,07 | 46,07 - 50,07 |

Method for production of honey powder being subject of this invention in its basic form comprises process steps of
(a) Mixing honey in the amounts specified in Table 1, preferably with at least one auxiliary encapsulation agent selected from a group consisting of maltodextrin, starch, gelatin, sodium caseinate, pectin, gum or a mixture thereof, in the amounts specified in Table 1;
(b) Measuring viscosity of mixture,
(c) Adding Distilled water to the mixture until reaching 15 - 20 mPas viscosity value,
(d) Applying homogenization process,
(e) Increasing temperature of mixture preferably to 45° C.,
(f) Drying at spray dryer and obtaining honey powder.

In an illustrative alternative of the method of invention before (a) process step, moisture, acid, conductivity, pH, colour, sensational, HMF; proline, diastase, invertase, sugar profile, C13 and C4, CAP, nitro furan, naphthalene streptomycin, sulphonamide, tetracycline group antibiotic residual, total phenolic, flavonoid, antioxidant capacity analysis are made for the honey to be used. Honey with high moisture content and suitable for analysis is weighed in the amounts specified in table 1 in the process step (a) and mixed with at least one auxiliary encapsulation agent in a pressurized container, preferably for 15 minutes. Before process step (b), natural protein of quantities given in Table 1 is preferably added and mixture is mixed at homogenizer 35 - 40° C., 2000 rpms for 2 hours. Then fruit juice concentrate of quantity given in table 1 is added and final mixture is mixed at homogenizer at 35 - 40° C., 2000 rpms, for 2 hours. Then respectively process steps of ‘b, c, d, e’ are applied. Mixture brought to temperature of 45° C. is dried in process step (f) at spray drier of input temperature 140 -150° C., output temperature 70 - 80° C. and feeding quantity 0.8-1 mL/s and thus honey powder is obtained.

In order to verify that honey powder obtained by means of above described method is of desired properties, HMF, proline, diastase, invertase, sugar profile, total phenolic, flavonoid, antioxidant capacity analysis are applied to the obtained honey powder. Products with proper analysis results are filled and packed for offer to the consumer.

1. A method for production of honey powder, comprising the process steps of:
(a) mixing honey with at least one auxiliary encapsulation agent selected from the group consisting of maltodextrin, starch, gelatin, sodium caseinate, pectin, gum or a mixture thereof;
(b) measuring viscosity of mixture;
(c) adding distilled water to the mixture until reaching a 15 - 20 mPas viscosity value;
(d) applying a homogenization process;
(e) increasing a temperature of the mixture; and
(f) drying at a spray dryer and obtaining honey powder.

2. The method according to claim 1, wherein before step (a) the honey undergoes the following analyses:
moisture, acid, conductivity, pH, color, sensory, HMF, proline, diastase, invertase, sugar profile, C13 and C4, CAP, nitrofuran, naphthalene streptomycin, sulfonamide, tetracycline group antibiotic residue, total phenolic, flavonoid, and antioxidant capacity.

3. The method according to claim 1, wherein in the process step (a), 250-750 g of honey is mixed with an amount of 250-750 g of auxiliary encapsulation agent selected from the group consisting of maltodextrin, starch, gelatin, sodium caseinate, pectin, gum or a mixture thereof.

4. The method according to claim 1, wherein the process step (a) takes place in a pressurized container.

5. The method according to claim 1, wherein in process step (a), the mixture is mixed for 15 minutes.

6. The method according to claim 1, wherein before the process step (b), a natural protein or fruit juice concentrate or their mixture is added to the mixture and the obtained mixture is mixed in the homogenizer.

7. The method according to claim 6, wherein the addition of natural protein is in an amount between 10-50 g.

8. The method according to claim 6, wherein the addition of fruit juice concentrate is in an amount of 10-50 g.

9. The method according to claim 7, wherein the obtained mixture is mixed at 35-40° C., 2000 revolutions for 2 hours in a homogenizer.

10. The method according to claim 1, wherein in the process step (f), an inlet temperature of the spray dryer is 140-150° C., an outlet temperature is 70-80° C., and a feed rate is 0.8-1 mL / s.

11. The method according to claim 1, wherein HMF, proline, diastase, invertase, sugar profile, total phenolic, flavonoid and antioxidant capacity analysis of the powder honey are obtained as a result of the process step (f).

12. A honey powder product comprising at least one auxiliary encapsulation agent selected from the group consisting of water soluble gum, maltodextrin, starch, gelatin, sodium caseinate pectin or a mixture thereof.

13. The product according to claim 12, wherein said at least one auxiliary encapsulation agent is water soluble gum or maltodextrin or a mixture thereof.

14. The product according to claim 12, wherein said at least one auxiliary encapsulation agent is mixture of 50% - 50.

15. The product according to claim 12, comprising natural protein, fruit juice concentrate or mixture thereof.

16. The use of the products according to claim 12 for food, healthy nutrition or apitherapy.

17. The method according to claim 1, wherein the process step (a) comprises mixing honey in an amount of 46.07 to 50.07% by weight with respect to the total weight, maltodextrin, starch, gelatin, sodium caseinate, pectin, gum or an amount of 46.07 to 50.07% by weight of the total weight mixing with at least one auxiliary encapsulation agent selected from the group consisting of the mixture thereof.

18. The product according to claim 17, wherein the amount of honey is 46.07%.

19. The product according to claim 17, wherein the amount of auxiliary encapsulation agent is 46.07%.

20. The method according to claim 6, wherein (b) before the process step, natural protein or fruit juice concentrate or a mixture thereof is added to the mixture in an amount of 0.92-2.92% by weight based on the total weight and the resulting mixture is mixed in the homogenizer.

21. The method according to claim 20, wherein the amount of natural protein or juice concentrate is 1.92%.

22. The method according to claim 1, wherein the process step (c) comprises increasing the temperature of the mixture to a maximum of 45° C.
